(19) United States
(12) Patent Application Publication
(10) Pub. No.: US 2025/0235162 A1
(43) Pub. Date: Jul. 24, 2025

(54) APPARATUS FOR DETERMINING A CARDIOVASCULAR RISK SCORE OF A USER

(71) Applicant: Aktiia SA, Neuchâtel (CH)

(72) Inventors: Josep Maria Solà I Carós, Cormondrèche (CH); David Perruchoud, Uster (CH); Meritxell Cortès, Zurich (CH); Olivier Grossenbacher, Neuchâtel (CH); Sibylle Fallet, Corcelles-près-Concise (CH)

(73) Assignee: Aktiia SA, Neuchâtel (CH)

(21) Appl. No.: 18/860,348

(22) PCT Filed: May 16, 2023
(86) PCT No.: PCT/IB2023/055037

§ 371 Date: Oct. 25, 2024

(30) Foreign Application Priority Data
May 16, 2022 (CH) 00588/2022

(51) Int. Cl. A61B 5/00; A61B 5/021

(52) U.S. Cl. CPC A61B 5/7275; A61B 5/021; A61B 5/7246

(57) ABSTRACT

A method comprising: providing an apparatus configured to measure a cardiovascular signal of a user: using the apparatus to measure the cardiovascular signal during a monitoring period having a duration of at least 24 hours: the monitoring period being subdivided into monitoring segments, each monitoring segment having a duration of 24 hours and comprising a plurality of measurement periods: determining a cardiovascular value for each measurement period: aggregating the cardiovascular values determined for a corresponding measurement period of each monitoring segment into a cardiovascular parameter cluster: constructing a circadian plot of 24 hours of the parameter clusters versus the corresponding measurement period: using the circadian plot to determine a plurality of physiological parameters of the user; and using the determined physiological parameters to calculate a cardiovascular risk score of the user.

### TECHNICAL DOMAIN

The present invention concerns an Apparatus and method for determining a cardiovascular risk score of a user.

### RELATED ART

Hypertension remains the leading risk factor for death worldwide. Despite its prevalence, success of blood pressure (BP) management efforts remains elusive, and part of the difficulty lies in the tool still used to diagnose, measure, and treat hypertension: the sphygmomanometer introduced by Samuel Siegfried Karl von Basch in 1867. In recent years, there has been an explosion of devices attempting to provide estimates of BP without a cuff, overcoming many limitations of cuff-based BP monitors. Unfortunately, the differences in underlying technologies between traditional BP cuffs and newer cuffless devices, as well as hesitancy of changing a well-implemented standard, still generate understandable skepticism about and reluctance to adopt cuffless BP monitors in clinical practice.

The scale of the problem of hypertension is difficult to comprehend-over 1.3 billion people worldwide are estimated to be hypertensive. Nevertheless, the current paradigm of diagnosis, treatment, and monitoring for hypertension (HTN) has resulted in poor rates of control. There is substantial risk in maintaining the status quo: HTN is and has been the single largest contributor to cardiovascular death and disease for over 40 years, is estimated to cost the US healthcare system $131 billion/year and is the leading preventable risk factor for premature death worldwide. Any change that may improve hypertension care, given the immense potential for benefit, must be explored fully.

The past two decades have witnessed a dramatic digital shift in healthcare, enabling a more robust, real-time, and practical exchange of data and information between patients and providers, primarily through widespread adoption of the EHR and accompanied by numerous digital health applications. Indeed, 20 years ago-seven years prior to the release of the first iPhone-the Institute of Medicine (IOM) authored a book in which the authors highlighted the potential of computer-aided (what is now termed digital health or mHealth) tools that assist in automating the transfer of clinical data to clinicians, both to improve clinical care and to further our understanding of disease.

Two decades later, technology is beginning to fulfill the loM's visionary insights and call to action. Cuffless BP technology promises to improve the diagnosis, treatment, and monitoring of hypertension, carrying the potential to benefit millions of hypertensive people.

Innovation has long been focused on treatment once a disease has been manifest, but now technology has afforded an opportunity to help prevent uncontrolled HTN and all its attendant risks. The commercialization of cuffless BP devices will allow us to solve many of the behavioral and practical challenges of treating a widespread chronic disease that has long languished in the background. The treatment of hypertension has been constrained by the limits of both in-office BP measurements and cuff-based home measurements. It is reasonable to expect that large scale adoption will allow realization of the benefits of cuffless BP devices, and in turn greater global hypertension control.

In 1948, one of the most important studies of cardiovascular risk was launched in Framingham, Massachusetts. There is little doubt of the seminal nature of the insights gained from the research over the subsequent three decades. As part of the study protocol, in-office measurements of BP were determined by the only available technology at the time-auscultation of the Korotkoff sounds to estimate systolic and diastolic BP. Subjects were seated with their backs against a chair, and measurements were taken in the left arm only. To this day, except for the notable shift from the manual mercury manometer to automated oscillometric devices, all major guidelines recommend that BP measurements are taken as originally described by the Framingham Study method.

After decades of cuff-measured targets within clinical trials and practice guidelines, this methodology of BP collection has been enshrined in collective medical thought as the standard for estimating BP. Furthermore, it has supported a hypothesis that an individual has a physiologically stable and predictable BP. Even expert consensus documents, such as the American Heart Association guidelines, state “it is generally agreed that conventional clinic readings, when made correctly, are a surrogate marker for a patient's true BP, which is conceived as the average over long periods of time, and which is thought to be the most important component of BP in determining its adverse effects.”

As the comprehension of hypertension has evolved, however, it is evident that BP continuously changes and adapts over 24 hours according to lifestyle, daily activities, medication treatment, physical/emotional stressors, and body position changes. Expert guidelines also suggest that in-office readings should be confirmed by out-of-office readings over subsequent weeks and months, implying that the true nature of an individual's BP patterns in daily life cannot be wholly estimated by relaxing for 5 minutes in a quiet, climate-controlled environment free of exercise, speaking, caffeine, noise, and with both feet flat on the floor.

Ambulatory BP monitors (ABPM) begin to show some of this variability, but usually over only 24 hours. Recently the development of continual cuffless BP devices enables, for the first time, a much more representative longitudinal depiction of an individual's BP.

The importance of out-of-office BP measurements is recognized in all major HTN guidelines for confirming office BP readings. ABPM has been considered the gold standard for out-of-office BP measurements.

However, ABPM remains woefully underutilized for a variety of reasons.

While ABPM may be the current recommended tool to monitor out-of-office BP, it is almost never used in the US. The percentage of Medicare beneficiaries—for whom the expected prevalence of HTN is estimated to be 50%—with ABPM claims was only ˜0.1% per year. In China, only 1.6% of primary care providers surveyed reported using ABPM to diagnose HTN. A simpler, cheaper, and more widely available solution for BP monitoring would be of significant benefit to providers and patients.

Home BP monitoring (HBPM) is also recommended by all major HTN guidelines as a critical adjunct to diagnosis, monitoring, and management for HTN. However, in practice it is difficult for patients to monitor their BP at home and send in meaningful data. In addition, patients need training to follow the same standard procedure of BP measurement as office BP measurements. The percentage of active HBPM in real-world patients is astonishingly low, despite the ready availability of relatively inexpensive home BP cuffs. Half of hypertensive patients report never checking their BP at home, 10% checked it less than once per month, and only 24% of hypertensive patients reported checking BP>1/week. Data demonstrate that while HBPM is routinely recommended by expert panels and consensus guidelines, it is not actually performed by most patients with HTN, and certainly not performed twice daily for at least seven days, as recommended.

There are many possible explanations for the marked gap between the recommendations and real-world practice. A study done in 2017 explored the barriers of primary care providers recommending HBPM to their patients. Over two thirds of respondents gave one or more of the following reasons as barriers to obtaining HBPM data:

Patients unable to complete HBPM due to low health literacy, time requirement.

intrusiveness of testing, requirement of a routine, and requirement to bring HBPM to the office.

Test results inaccurate due to patient noncompliance with HBPM protocol (e.g. incorrect cuff size, poor timing of BP readings, failure to record readings, “cherry-picking” normal BP readings to show physicians.

Inaccurate results due to patient factors such as body habitus.
Test results or cuff inflation could increase patient anxiety and hence accuracy.

### SUMMARY

Blood Pressure (BP) is known to change over time and follow a circadian rhythm. It typically exhibits higher systolic and diastolic values during day-time and decreases during night-time and sleep. This nocturnal drop of blood pressure is commonly referred to as the “night-dipping”. But the exact profile of this night-dipping can widely vary across individual, and even slowly shift over time. Some parameters of this profile, such as the amplitude of the night-dipping, or the slope of the morning rise (also referred to as “morning surge”) is clinically relevant, as it correlates with different cardiovascular risk factors.

The present disclosure concerns a method for determining a cardiovascular risk score of a user comprising:
providing an apparatus configured to measure at least a cardiovascular signal of a user;
measuring said at least a cardiovascular signal during a monitoring period having a duration of at least 24 hours;
the monitoring period being subdivided into at least one monitoring segment, wherein said at least one monitoring segment has a duration of 24 hours and comprises a plurality of measurement periods;
determining a cardiovascular value for each measurement period of the plurality of measurement periods within said at least one monitoring segment;
aggregating the cardiovascular values determined for a given measurement period of each said at least one monitoring segment into a cardiovascular parameter cluster;
constructing a circadian plot of 24 hours of the cardiovascular parameter clusters versus the corresponding measurement period for each said at least one monitoring segment;
calculating a plurality of physiological parameters of the user from the circadian plot; and
calculating a cardiovascular risk score of the user from the determined physiological parameters.

The present disclosure further concerns an apparatus for determining a cardiovascular risk score of a user.

The present disclosure further concerns a computer program comprising instructions which, when the program is executed by a computer, cause the computer to carry out the steps of the method.

### SHORT DESCRIPTION OF THE DRAWINGS

Exemplar embodiments are disclosed in the description and illustrated by the drawings in which:

FIGS. 1a-1d illustrate an apparatus and a method for calculating a cardiovascular risk score of a user;

FIGS. 2 to 6 show examples of possible fitting a models, namely a trapeze model (FIG. 2), a rectangular model (FIG. 3), a gaussian model (FIG. 4), a skewed gaussian model (FIG. 5), and a skewed flattened gaussian model (FIG. 6);

FIG. 7 reports the population comparison of daytime BP averages by the apparatus against the HBPM measurements obtained during the initialization of the device on the same day;

FIGS. 8a and 8b illustrate the poor intra-subject reproducibility of ABPM exams: because the circadian BP excursions of an individual varies over time, by choosing arbitrary day(s) of measurement, an ABPM exam might generate a phenotype that misrepresents the underlying BP phenotype of the patient;

FIG. 9. Comparison of the estimation of nocturnal dipping as measured by ABPM and by the apparatus;

FIGS. 10a to 10j show the new generation of dynamic BP control metrics that can be generated from the data captured from the apparatus;

FIG. 11 shows the apparatus for determining a cardiovascular risk score of a user, according to an embodiment; and

FIG. 12 is a cross section view of the apparatus, according to an embodiment.

### DESCRIPTION

The present disclosure describes a method and apparatus to build a model of the BP circadian rhythm for a specific user, given his/her history of BP measurements over a period of time and to derive from this model a set of relevant parameters that can be used assess the cardiovascular risk score of the user and to further communicate this cardiovascular risk score to the user.

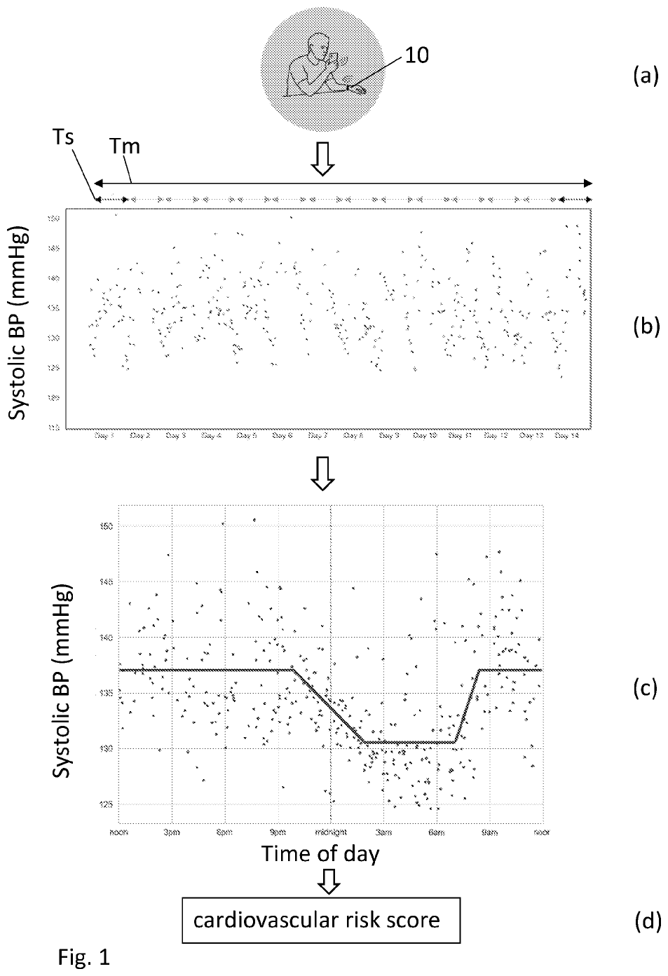

In particular (see FIGS. 1a to 1b), a cardiovascular signal of the user is measured by using an apparatus 10 configured to measure such cardiovascular signal, during a monitoring period Tm (FIG. 1b). The monitoring period Tm has a duration of at least 24 hours and is subdivided into a plurality of measurement periods (not show). For each measurement period, a cardiovascular parameter of the user is determined.

Each cardiovascular parameter determined for a corresponding measurement period is aggregated into a cardiovascular parameter cluster.

A circadian plot of the cardiovascular parameter clusters versus the corresponding measurement period can then be constructed.

For a given user, the 24/7 (24 hours a day, 7 days a week) systolic and diastolic values of BP for a consecutive period of time (for example two weeks) are aggregated together. On the temporality of each measurement, the exact day of each measurements is ignored, and only the time information (hours and minutes) is conserved. This process coalesces all of the BP measurements (systolic or diastolic, separately) of the whole period of investigation over a single 24-hour period, centered around midnight. For each measurement period of one hour (e.g. from 7:00 am to 7:59 am), any BP measurement outside of the limits defined as the median ±2×IQR (InterQuartile Range) for said period are discarded prior to further analysis (outlier rejection).

Over the remaining data, a constrained piece-wise linear model is fitted via Least-Square optimization. The model is constrained in such a way that the BP profile is restricted to a constant during the nocturnal dipping, and to a different constant during day-time, and allows for two linear transitions (ramps) between those two states.

Once built, this model provides a set of six physiological parameters, uniquely describing the BP profile of the individual (see FIGS. 1b), namely:
diurnal BP value (Y₀),
absolute night-dipping amplitude (ampl),
temporality of the dipping start (X0),
duration of the pre-nocturnal ramp (dl),
duration of the dipping plateau (nl), and
duration of the post-nocturnal ramp (al).

The physiological parameters can be further combined into additional clinically-relevant parameters, such as (non-exhaustive):
nocturnal BP value,
relative night-dipping amplitude,
complete night-dipping duration,
time in target range (TTR), or
slope of the morning surge.

The calculated physiological parameters can further allow to the determine the blood pressure phenotype of the user, including classification such as: true normotension, white-coat hypertension, masked hypertension, sustained hypertension, hypotension, night-dipping, night-raising.

The physiological parameters and/or the clinically-relevant parameters and/or blood pressure phenotypes can be further combined into additional scores of cardiovascular risk.

In an embodiment illustrated in FIGS. 1a to 1d, a method for calculating a cardiovascular risk score of a user comprises:
providing an apparatus 10 configured to measure at least a cardiovascular signal of a user (FIG. 1a);
using the apparatus 10 to measure said at least a cardiovascular signal during a monitoring period Tm having a duration of at least 24 hours (FIG. 1b);
the monitoring period Tm being subdivided into at least one monitoring segments Ts, wherein said at least one monitoring segment Ts has a duration of 24 (twenty-four) hours and comprising a plurality of measurement periods;
determining a cardiovascular value for each measurement period of the plurality of measurement periods within said at least one monitoring segment Ts;
aggregating the cardiovascular values determined for a given measurement period of each said at least one monitoring segment Ts into a cardiovascular parameter cluster;
constructing a circadian plot of 24 (twenty-four) hours of the parameter clusters versus the corresponding measurement period for each said at least one monitoring segment Ts;
using the circadian plot to determine a plurality of physiological parameters of the user (FIG. 1c); and
using the determined physiological parameters to calculate a cardiovascular risk score of the user (FIG. 1d).

The measurement period corresponds to the time period when the cardiovascular signal is measured by the apparatus 10.

In FIGS. 1a to 1d, FIG. 1a shows the apparatus 10 represented as cuffless blood pressure optical sensor at the wrist, FIG. 1b cardiovascular values (in this case, the BP values) calculated from the cardiovascular signal (in this case, a PPG signal, not shown) provided to the apparatus 10, and

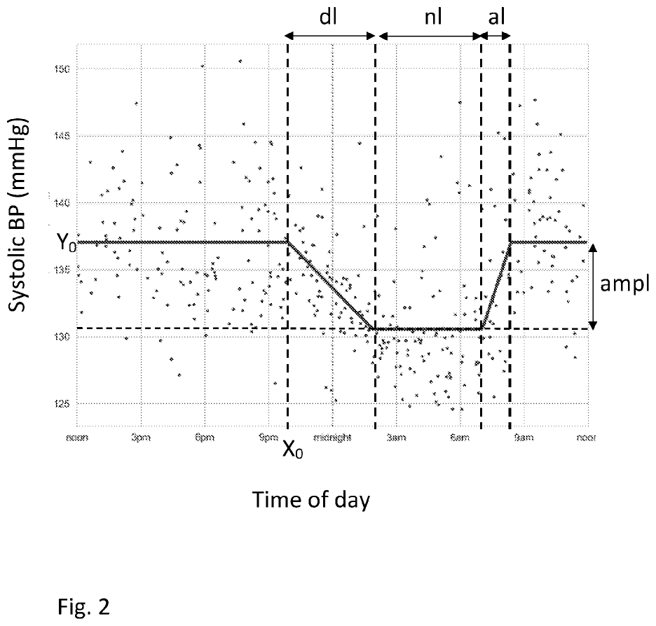

FIG. 1c shows aggregation of cardiovascular values of each monitoring segment Ts for the entire monitoring period Tm (see FIG. 2) into a cardiovascular parameter cluster, and construction of a circadian plot of 24 hours, and fitting of a model to the circadian plot, determination of a plurality of physiological parameters. From the plurality of physiological parameters a cardiovascular risk score of the user is calculated (FIG. 1d).

In one aspect, the monitoring period Tm has a duration of 48 hours, seven days, one month, or one year. Note that, in the case the monitoring period Tm has a duration of 24 hours, its duration is equal to the duration of the monitoring segment Ts.

In another aspect, the measurement period can have a duration of at least 10 seconds or 30 seconds, namely a duration of a duration, 1 minute, 5 minutes, one hour, two hours, four hours, or six hours.

In the example of FIG. 1b, the monitoring period Tm is more than 24 hours. For example the monitoring period Tm corresponds to two weeks (14 days). In that case, the monitoring period Tm comprises more than one monitoring segment Ts (14 monitoring segments of 24-hour duration for the 2-week monitoring period Tm).

Each 24-hour monitoring segment Ts comprises a plurality of measurement periods. For example, the monitoring segment Ts comprises twenty-four measurement periods of one hour duration. Thus, a plurality of cardiovascular values are calculated for each measurement period corresponding to a time of the day (twenty-four cardiovascular values are calculated for each time of the day).

Thus, a plurality of cardiovascular values are aggregated into a cardiovascular parameter cluster for each time of the day that correspond to each measurement period (see FIG. 1c).

In one aspect, the cardiovascular signal can be measured separately during working days and non-working days.

The cardiovascular signal can comprise any one of a measure of physical activity, measures of sleep quantity and quality, and values derived from an electrocardiogramal, a value derived from a photoplethysmographic signal, a value derived from a bioimpedance signal, a value derived from an ultra-sound signal, or a value derived from any arterial pulsatility signal such as pulse pressure.

The cardiovascular value can be calculated from the cardiovascular signal measured during a measurement period. Calculating the cardiovascular value can comprise identifying a plurality of pulses of the cardiovascular signal. Calculating the cardiovascular value can further comprise determining at least one feature of the identified pulses, and calculating the cardiovascular value based on said at least one feature. For example, a method for calculating cardiovascular values is presented in European patent publication EP3226758. In other embodiments, the cardiovascular value can also calculated without identifying a plurality of consecutive pulses of the cardiovascular signal and applying non-parametric algorithms or machine-learning algorithms.

The step of determining a cardiovascular value can comprise calculating a cardiovascular value from each cardiovascular signal. The cardiovascular value can comprise any one of a systolic BP value, a diastolic BP value, a mean BP value, a heart rate value, or a blood glucose value.

The cardiovascular value can further comprise any one of a pulse pressure, central pulse wave velocity, peripheral pulse wave velocity, arterial stiffness, aortic pulse transit time, augmentation index, stroke volume, stroke volume variations, pulse pressure variations, cardiac output, systemic vascular resistance, venous pressure, systemic hemodynamic parameters, pulmonary hemodynamic parameters, cerebral hemodynamic parameters, heart rate, heart rate variability, inter-beat intervals, arrhythmias detection, ejection duration, SpO2, SpHb, SpMet, SpCO, respiratory rate, tidal volume, and general cardiovascular and health indexes.

The step of aggregating the cardiovascular values can comprise grouping the cardiovascular values that are determined by the apparatus 10 during a given measurement period into a cardiovascular parameter cluster (see FIGS. 1b and 1c).

The step of constructing a circadian plot of 24 hours can comprise plotting the cardiovascular parameter cluster calculated for each measurement period as a function of the time in the day (a period of 24 hours), for example from midnight to midnight (see FIG. 1c).

In one aspect, the cardiovascular parameter clusters can have a duration of 10 seconds, 30 seconds, 1 minute, 5 minutes, 10 minutes, or 30 minutes.

The step of using the circadian plot can comprise calculating a plurality of physiological parameters of the user from the circadian plot.

In one aspect, the step of using the circadian plot can comprise calculating, for each cardiovascular parameter cluster, a cardiovascular representative value of the cardiovascular values aggregated into the cardiovascular parameter cluster.

In another aspect, the step of using the circadian plot can comprises calculating the cardiovascular representative value comprises classifying the cardiovascular parameters as inliers or as outliers, and calculating the cardiovascular representative value using only the inlier cardiovascular parameters.

In one aspect, calculating the cardiovascular representative value can comprise classifying the cardiovascular parameters as inliers or as outliers. The cardiovascular parameters can be weighted according to their probability of being inlier or outlier.

In one aspect, determining the plurality of physiological parameters of the user can comprise a step of fitting a model to the circadian plot, the physiological parameters corresponding to parameters of the model.

The model can be one of a linear model fitted via a least-square optimization, a non-linear model, a constrained model, an unconstrained model.

In one aspect, the constrained model can comprise temporal constrains on the duration of a physiological parameter (for instance, a blood pressure night dipping plateau cannot be longer than 12 hours, or a duration of the pre-nocturnal ramp cannot be longer than the duration of the dipping plateau), or can comprise amplitude constraints on a physiological parameter (for instance, a day time blood pressure value cannot be higher than 200 mmHg, or the SBP morning surge cannot be larger than 50 mmHg/hour).

As shown in FIG. 2, the physiological parameters can include any one of diurnal BP value (Y0), absolute night-dipping amplitude (ampl), temporality of the dipping start (X0), duration of the pre-nocturnal ramp (dl), duration of the dipping plateau (nl), or duration of the post-nocturnal ramp (al).

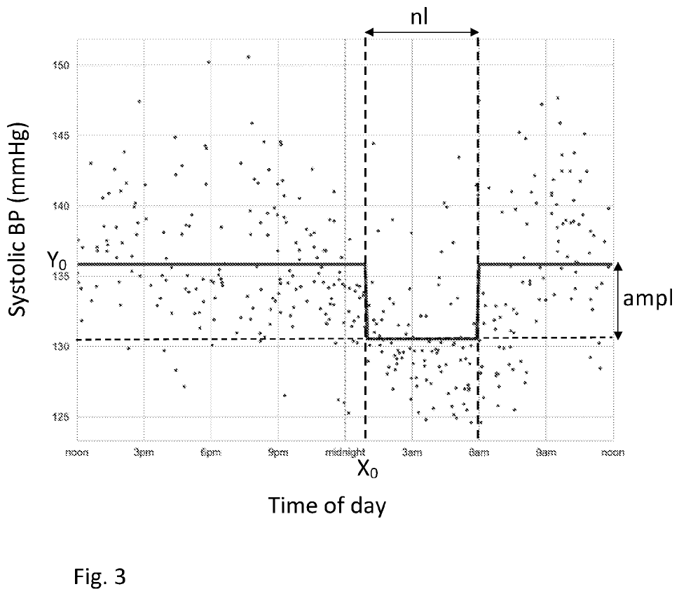

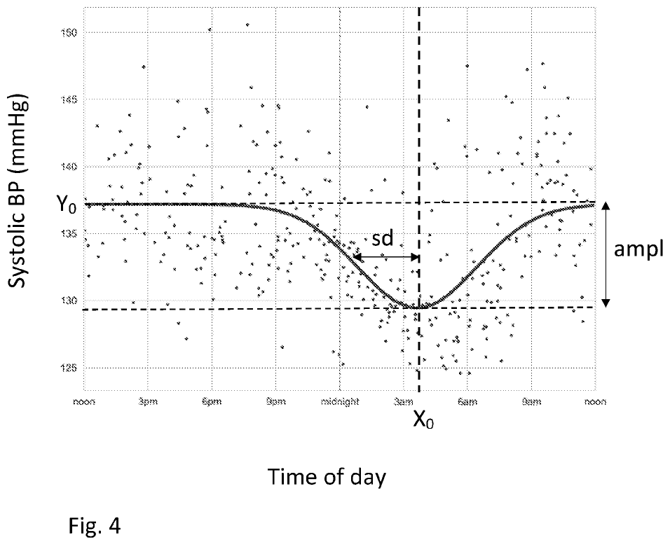

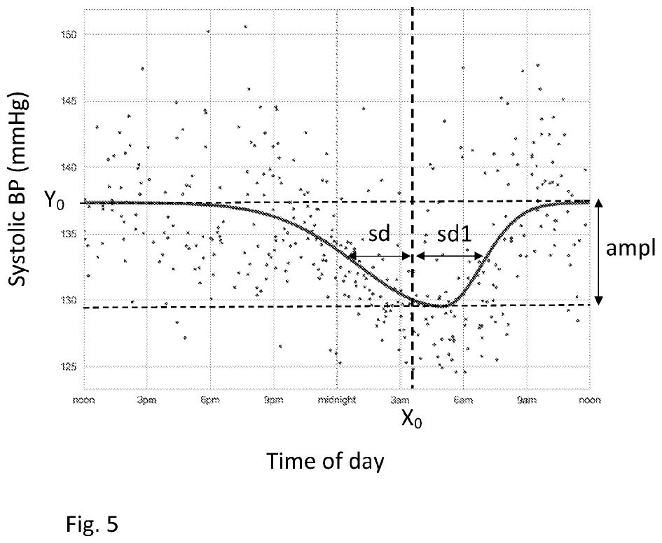

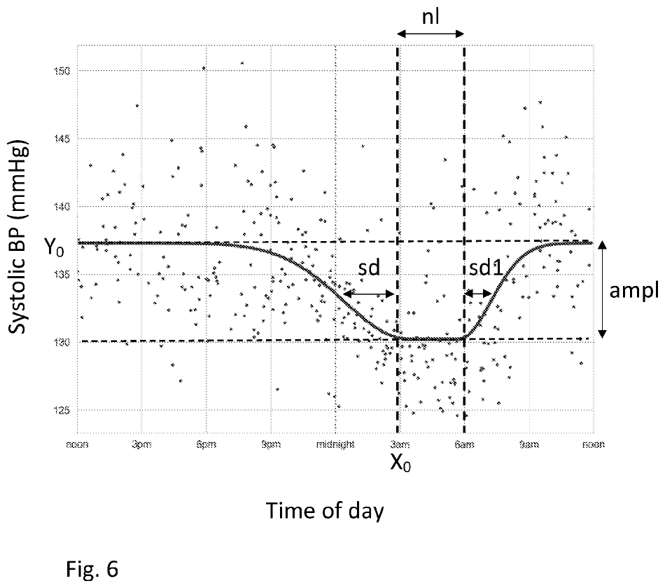

FIGS. 2 to 6 show examples of possible fitting a models, namely a trapeze model including six parameters Yₒ, Xₒ, ampl, dl, nl, al (FIG. 2), a rectangular model including four parameters Yₒ, Xₒ, ampl, nl (FIG. 3), a gaussian model including four parameters Yₒ, Xₒ, sd, ampl (FIG. 4), a skewed gaussian model including five parameters Yₒ, Xₒ, sdₒ, sd₁, ampl (FIG. 5), and a skewed flattened gaussian model including six parameters Yₒ, Xₒ, sdₒ, sd₁, ampl, nl (FIG. 6).

The physiological parameters can include a difference of a physiological parameter when calculated during workweek and the weekend, or more generally, a difference of a physiological parameter when calculated during working days and non-working days.

The physiological parameters can further include temporal dynamics of any of the physiological parameters. The temporal dynamics of the physiological parameter can include day-to-day variability of the parameter, spread of the parameter over a certain number of days, trend of the parameter over a certain number of days, or number of days for which the parameter is above or below a threshold.

In one aspect, the model further uses non-physiological parameters. Non-physiological parameters include parameters such as geo-localization of the user including altitude, weather forecast and observations, heat waves, cold waves, travelling information, pollution information, public health information including infectious disease status at the location of the user, time of the year, allergenic information, daylight patterns, social information, education level, familial situation (marital status, number and age of children), financial information, political interest and views, professional situation including level of responsibility, type of contract, regularity of working hours, information from a calendar including working/non-working days and holidays, working load, working agenda, dietary patterns exercise/activity patterns, level of sedentism, leisure information including consumption of caffein, alcohol, drugs, medications (not limited to anti-hypertensive), health situation of the user and of close relatives, type and size of accommodation, homeowner or renter pets, use of social media, religious practice, questionnaires on mood and general being.

The method can further comprise combining at least two of the physiological parameters to obtain one or a plurality of relevant physiological parameter. The combination can include adding, multiplying, or dividing at least two physiological parameters. Such combination can further include calculating a correlation coefficient or a synchronization coefficient of two or more physiological parameters.

The relevant physiological parameter can include any one of: daytime BP value, nocturnal BP value, relative night-dipping amplitude, complete night-dipping duration, time in target range (TTR), BP variability patterns, slope of the morning surge, nighttime SBP, nighttime DBP, nighttime HR, SBP dip, DBP dip, HR dip, SBP morning surge, DBP morning surge, HR morning surge, SBP dipping duration, DBP dipping duration, HR dipping duration, SBP/DBP/HR synchronization, responses to types of medications, BP medication adherence, patient engagement measures, or responses to lifestyle interventions.

In one aspect, the relevant physiological parameter can include a blood pressure phenotype of the user.

In one aspect, the relevant physiological parameters include any one of: true normotension, white-coat hypertension, masked hypertension, sustained hypertension, hypotension, night-dipping, night-raising, or phenotypes that predict responses to specific medications or therapies (such as renal denervation).

The step of using the determined physiological parameters to calculate a cardiovascular risk score of the user can comprise calculating a cardiovascular risk score of the user from the determined physiological parameters.

In one aspect, calculating the cardiovascular risk score of the user comprises using user data.

The user data can include any one of: as age, weight, height, gender, ethnicity, lipid levels, diabetes status, smoking, CT calcium (Agatston score), family history, genetic markers of risk, actigraphy information, workout information, dietary information, stress level, general feeling, hormonal data, menstrual cycle information, medication intake, working-day/week-end information, seasonal information, sleep quality information, go to bed patterns or any of the parameters used in the calculation of cardiovascular risk score in clinical guidelines such as the ACC/AHA guidelines, the ESC guidelines, or the MESA database. The user data can also be any of the non-physiological parameters.

The user data can be manually provided by the user, or automatically integrated from an external system.

The cardiovascular risk score can be any of 10-year (ten-year) risk of cardiovascular disease, 10-year risk of heart disease, 10-year risk of stroke or any other clinically relevant cardiovascular risk score.

In one aspect, the cardiovascular parameter can be at least any of a blood pressure value, a heart rate value, a cardiac output value, a blood glucose value, a measure of physical activity, or measures of sleep quantity and quality.

Also disclosed is an apparatus for determining a cardiovascular risk score of a user.

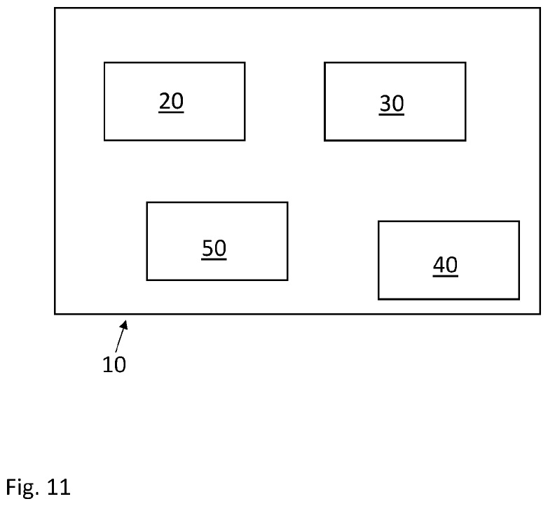

The apparatus 10 (see FIG. 11) comprises a measuring module 20 configured to measure a cardiovascular signal of the user during a monitoring period Tm having a duration of at least 24 (twenty four) hours, wherein the monitoring period Tm is subdivided into monitoring segments Ts, each monitoring segment Ts having a duration of 24 (twenty four) hours and comprising a plurality of measurement periods.

The apparatus 10 further comprises a processor 30 configured to determine a cardiovascular value from the measured cardiovascular signal for each measurement period, aggregate the cardiovascular values determined for a corresponding measurement period of each monitoring segment Ts into a cardiovascular parameter cluster, construct a circadian plot of 24 (twenty four) hours of the parameter clusters versus the corresponding measurement period, using the circadian plot to determine a plurality of physiological parameters of the user, and using the determined physiological parameters to calculate a cardiovascular risk score of the user.

The apparatus further comprises an interface 40 to display and/or transmit the calculated cardiovascular risk score. The interface 40 is operatively connected to the processor 30.

The processor 30 can be configured to perform the steps of determining a cardiovascular value, aggregating the cardiovascular values, constructing a circadian plot of 24 hours, calculating a plurality of physiological parameters of the user, and calculating a cardiovascular risk score of the user.

The apparatus 10 can be operatively connected to a wired or wireless communication circuit, the latter possibly including WiFi or Bluetooth or cellular supports. The apparatus 10 can be further operatively connected to a memory.

The interface 40 can comprise an application on a smartphone, a tablet, a computer, a smartwatch or any portable device.

The interface 40 can be configured to produce an external signal destined to the user, for example to provide guidance on how to optimize the calculated risk of the user by suggesting lifestyle, medication or treatment modifications.

The interface 40 can also be configured to input manually or automatically non-physiological data or user data.

The interface can be located close to the user or remotely from the user.

In one aspect, the measuring module 20 can be configured to measure the cardiovascular signal automatically without user interaction.

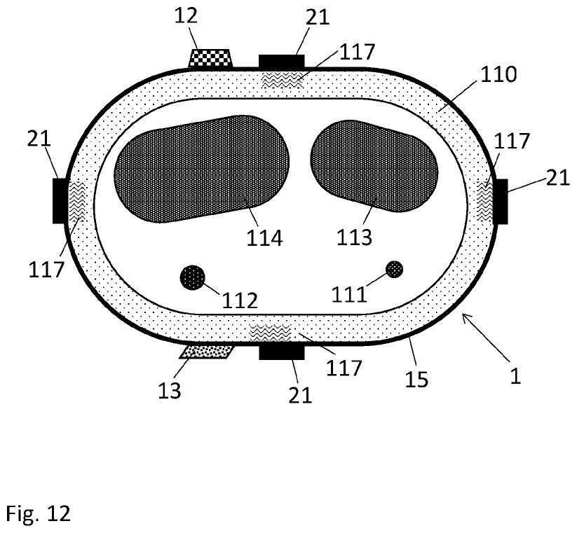

The measuring module 20 can comprises any one of an arterial pulsatility sensor such as galvanic skin response (GSR) sensor array, a bioimpedance (BioZ) sensor array, an electrocardiogramaor (ECG), a sensor based on radio frequency (RF) detection, a radar sensor, a mechanical sensor, a pressure sensor, an invasive sensor, an intra-arterial sensor, a minimal invasive sensor, a subcutaneous sensor, a tonometer, a strain sensor, a plethysmographic sensor, a microphone, an ultrasound sensor, a capacitive sensor, an electromagnetic sensor, a Raman sensor, or any sensor capable of measuring a pulsatility signal either from the capillary bed of the skin or from any other section of the arterial tree. The cardiovascular signal measured by the apparatus 10, via the measuring module 20, can thus correspond to the signal measured by the The apparatus 10 can comprise a wearable device. A possible configuration of the apparatus 10 being a wearable device is illustrated in the cross section view of FIG. 12. The apparatus 10 may include a wristband 15 comprising the measuring module 20. The measuring module 20 can comprise at least one pulsatility sensing unit 21. For example, the measuring module 20 comprises four pulsatility sensing units 21 distributed along the inner side of the wristband 15 periphery such as to be in contact with the user's wrist skin when the apparatus 10 is worn. Other arrangements of the pulsatility sensing units 21 on the wristband 15 are possible.

In one embodiment, the pulsatility sensing units 21 may comprise a photoplethysmograph (PPG) sensor array that may measure arterial pulsation, arterial diameter, blood flow and/or blood content. In that case, the cardiovascular signal is a photoplethysmographic signal. In this embodiment, the pulsatility sensing unit 21 may be arranged on the wristband 15 so that the optical sensor array 21 straddles or otherwise addresses an artery, such as the ulnar artery 111, in the vicinity of the ulna bone 113, or radial artery 112, in the vicinity of the radius bone 114 (as shown in FIG. 12) or any arterial vascular bed 117 of the skin of the wrist.

In an embodiment, the apparatus 10 further comprises a triggering module 50 (see FIG. 11) configured to initiate or stop measurement period by the measuring module 20.

The triggering module 50 can control the measuring module 20 according to a trigger parameter. The trigger parameter can be specific to a user. Examples of trigger parameter can include a trigger signal such as a motion signal representative a user's movement. Such motion signal can be measured by using a motion sensor 60 placed on the user, for example on the apparatus 10. The motion sensor 60 can include any one of an inertial measurement unit (IMU), an accelerometer, a gyroscope, magnetometer, or a combination of these devices.

In one aspect, the cardiovascular parameter is a blood pressure (BP) value.

The blood pressure value can be measured by using a cuff-based BP measurement technique and/or by using an optical sensor. The blood pressure value is measured at the wrist.

The blood pressure value comprises at least one of Systolic BP, Diastolic BP or Mean BP.

The apparatus 10 disclosed herein allows for delivering blood pressure value in a continuous fashion, for example during day and night.

The apparatus 10 disclosed herein corresponds to a cuffless BP monitoring device. A cuffless BP device has the potential to solve many practical and behavioral issues, to overcome barriers to recommended routine monitoring of BP, and to obtain significantly more BP data compared to traditional methods. The ability to collect continual BP readings at home, out of the office, during daily activities, and while sleeping, over periods of weeks, months, and years, gives patients and providers a far more complete assessment of BP than intermittent checks in a controlled position and environment, which provide physicians and patients only glimpses of the complete representation of BP.

The ability to obtain continuous BP readings out of the office and

during day and night has been traditionally limited because available monitoring technologies required the inflation of a cuff for each measurement. Devices that do not require inflation of a cuff have can overcome most of the limitations inherent in traditional BP cuff monitors.

The apparatus 10 allows for non-invasively determines the BP of an individual without creating any arterial occlusion.

The apparatus 10 can be placed in body locations such as the wrist, fingertip, chest, ear, forehead, or a combination.

The apparatus 10 provides an indirect estimation of BP that relies on the analysis of the arterial pulses at one or more body location(s) with a sensor that applies no pressure to that location. The apparatus 10 does not provide a direct pressure measurement, but a quantity that a computer program calculates from the analysis of the waveform of a pressure pulse which is mapped to a BP value typically following an initialization phase. Several sensor technologies (sensing units 21) are currently used to capture the waveforms of pressure pulses ranging from optical sensors (assessing the pulsatility of skin arterioles via reflection or transmission photo-plethysmographic sensors), camera sensors (assessing the pulsatility of skin arterioles via reflection video-based photo-plethysmography), biopotential sensors (assessing different electro-magnetic signatures of the cardiac activity, or assessing arterial pulsatility from impedance plethysmography signals at different body locations), radar sensors (assessing arterial pulsatility at different body locations from radar reflections) and tonometric sensors (assessing pulsatility of superficial arteries by sensing displacements of the skin). Depending on the number of body locations on which a pressure pulse is captured on the patient body, the analysis of the waveforms is performed based either on pulse wave velocity algorithms (typically when at least two body locations are involved) or on pulse wave analysis algorithms (typically when one single body location is involved). Because no pressure measurement is involved in the assessment of such pulsatility waveforms, most cuffless BP monitor still require an initialization procedure that involves the use of an oscillometric device to provide information in “mmHg”.

A major appealing feature of the apparatus 10 is the potential to provide significantly more BP data points. The ability to collect continual BP readings at home, out of the office, in daily life, and at night and while sleeping, over periods of days to years, gives patients and providers a far more representative assessment of BP than occasional cuff estimates. The snapshots of BP measured in-office or by HBPM at one point in time represent only a fraction of the full dynamic data set of BPs. Without these data, physicians and patients are essentially blind to the true nature of BP.

To illustrate these limitations, real-world systolic BP (SBP) data were recorded on a male subject using the apparatus 10 over two months. The data demonstrate the stark disparity between simulated in-office readings, occasional home BP monitoring, and ambulatory BP monitoring. The in-office estimate suggests a significantly higher absolute SBP value than the average and does not capture longitudinal BP data, demonstrated most clearly and commonly in white-coat and masked HTN syndromes (up to 40% of individuals). Home BP estimates of SBP-when performed routinely—may correlate with overall averages, but do not capture the daily and circadian variability. Finally, an ambulatory BP monitor reveals information during only a narrow (24- or 48-hour) period. All three traditional methods of BP estimates represent only glimpses into the dynamic BP, which is demonstrated very well by the apparatus 10.

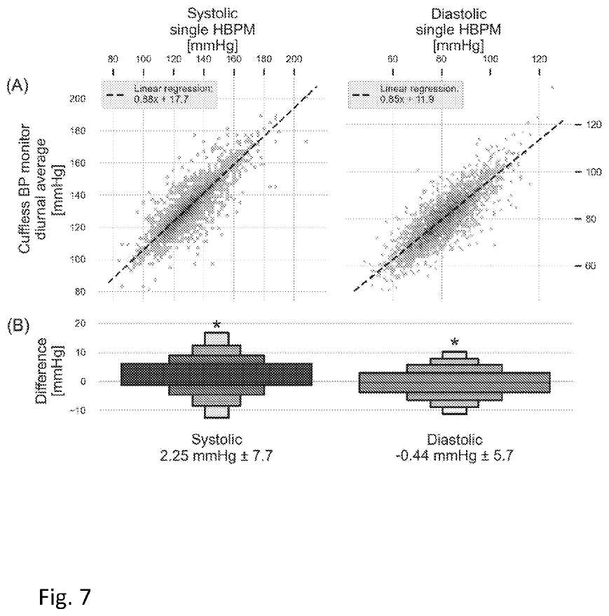

Compared to other existing technologies, the apparatus 10, can provide markedly more BP readings, demonstrate better the variability of BP, and provide nighttime BP measures, all of which have meaningful clinical implications. A clinician may wonder, however, how the daytime average BP provided by the apparatus 10 compares to an HBPM reading performed on the same day? To answer this question, the anonymized data from 2,928 users of the apparatus 10 was analyzed offline (FIGS. 7a and 7b). The analysis compared diurnal BP data (between 8 am and 8 pm) measured by the apparatus 10 on the day of the initialization procedure, against the brachial cuff BP measurement (HBPM) obtained during same initialization procedure. The analysis was repeated both for systolic and diastolic BP. In both cases, a paired T-test showed that the difference in measurements with the two modalities was statistically significant (both p<0.001), although the differences (SBP of 2.25 mmHg and DBP of 0.44 mmHg) were below the resolution and error margin of any automated oscillometric BP monitor.

FIGS. 7a and 7b shows population comparison of daytime BP averages by apparatus 10 against the HBPM measurements obtained during the initialization of the device on the same day. FIG. 7a presents one data point from each of the 2,928 users of the apparatus 10 for systolic and diastolic BP. On the X axis is the single measure from an HBPM read during the initialization of the device, compared on the Y axis with the concurrent diurnal (8 am-8 pm) average BP measured by the apparatus 10. Left, SBP. Right, DBP. The dotted line is calculated with a Huber linear regression. The letter-value plot on FIG. 7b depicts the distribution of the pairwise differences between the values measured by the apparatus 10 and the corresponding HBPM values, for systolic and diastolic BP. Mean and standard deviation of each distribution are presented on the bottom. Asterisks denotes statistically significant differences between the HBPM and measurements obtained by the apparatus 10 (both p<0.001).

Using real-world data from over 2,000 patients using the apparatus 10, the distinct advantages of a markedly richer BP data set, the ability to measure nocturnal BP longitudinally, and the automated, passive nature of the device are striking when compared to traditional monitoring (Table 1). Furthermore, the systematic differences of daytime BP averages of the apparatus 10 as compared to daytime HBPM readings across this population are small and within an acceptable margin of error. In summary, the apparatus 10 has tremendous potential to greatly improve the ability to monitor BP in the ambulatory setting.

TABLE 1
Guidance for the interpretation of daytime averages obtained from the
apparatus 10when compared to daytime averages obtained from HBPM.

| Criterion | HBPM | Apparatus 10 |
|---|---|---|
| Triggering of | Manually triggered by the patient | Automated, imperceptible by |
| readings | | the patient |
| Frequency of | As low as once a month to twice | On average, approximately |
| readings | daily (highly compliant patient) | one reading per hour (avg |
| | | from 4,887 users of the |
| | | apparatus 10) |
| Conditions of | Patient is sitting and relaxed with | Anytime the patient is quiet |
| measurement | the arm at the heart level | or performs no important |
| during daytime | | movement (motion tolerance |
| | | might vary across devices) |
| | | with no control of body and |
| | | arm position |
| Availability of | No | Yes |
| nighttime readings | | |
| Feasibility of long- | Yes, but only for patients with high | Yes, even for patients with |
| term monitoring | compliance | reduced compliance |

| | |
|---|---|
| Systematic | Daytime SBP average is similar (2.2 mm Hg higher) to same-day |
| difference of | HBPM |
| daytime BP | Daytime DBP average is similar (0.44 mm Hg lower) to same-day |
| averages (Cuffless | HBPM |
| vs. HBPM) | |

ABPM remains the recommended modality when more complete analysis of a patient's BP pattern is required for the diagnosis/monitoring of

HTN, despite its low utilization in clinical practice (see previous sections). While ABPM is the only currently recommended modality able to obtain day and night BP readings, its infrequent use raises questions of reproducibility. Circadian excursions of BP are known to be dynamic, and by arbitrarily picking a short monitoring period (24 or 48 hours), a clinician may obtain data representative of only a narrow sliver of the overall BP.

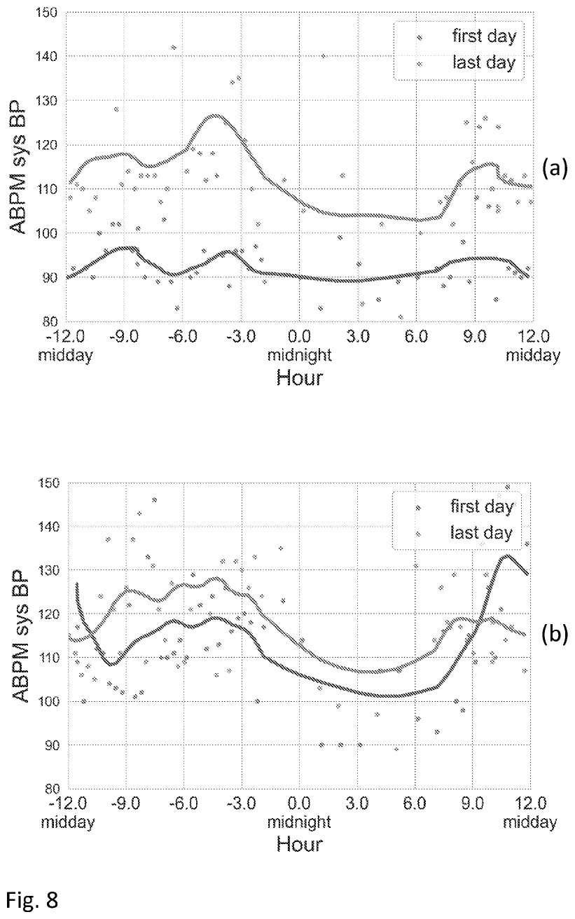

To further illustrate the reproducibility problem of ABPM, FIGS. 8a and 8b show two examples of repeated ABPM recordings from a running clinical trial (lower panel). The meta-analysis of 35 observational studies demonstrates that for ⅓ of the patients, the classification of dipper/non-dipper status is not reproducible for two consecutive ABPM nights (e.g. on the first night a patient is classified as a dipper, but on the following night is classified as a non-dipper), and that the observed differences of nocturnal averages of SBP and DBP between two consecutive nights can vary between-19.6 and 21.3 mmHg, and −11.3 and 12.3 mmHg respectively. These data confirm the reproducibility of ABPM on assessing intra-individual dipping status and daytime and nighttime BP values is limited. FIGS. 8a and 8b provide ABPM recordings from selected patients of an ABPM study, FIG. 8a presenting very poor intra-subject reproducibility, and FIG. 8b presenting better reproducibility on the measured night dip and daytime/nighttime averages of BP.

Given the documented poor reproducibility of ABPMs, the apparatus 10 may overcome the arbitrary nature of cuff-based ABPMs by exploiting the ability of the apparatus 10 to generate voluminous data over the long-term. However, comparison of data between the two modalities requires consideration of two factors. First, the apparatus 10 fundamentally measures BP differently than those measured by traditional oscillometric ABPMs. Second, frequency and period of ABPM measurements (e.g. once every 20 minutes) differs from that of the apparatus 10 (e.g. when the user is still for long enough). This difference captures BP during different daily activities, with ABPM largely capturing more readings during physically active periods than the apparatus 10. These two differences (technological and triggering timing) are thus expected to generate daytime and nighttime averages of BP that might differ between modalities.

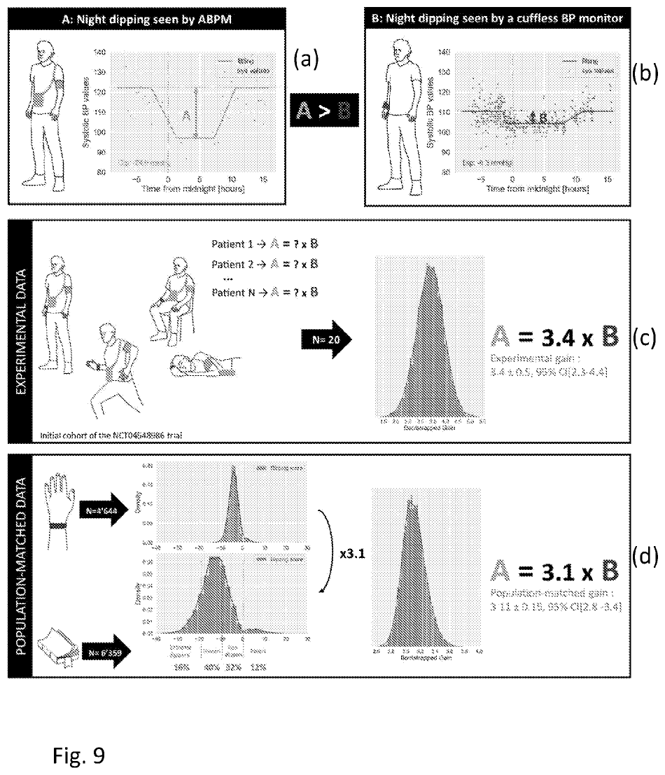

FIGS. 9a to 9d provides a first glimpse on the systematic differences observed between ABPM and the apparatus 10 when estimating night dipping status of patients.

In FIGS. 9a and 9b illustrate differences in estimated SBP dips on a patient of the NCT04548986 trial. FIG. 9c shows a systematic factor of 3.4 across the initial cohort of patients of the same trial. FIG. 9d shows a systematic factor of 3.1 across N=4,644 users of the apparatus 10 when matching the phenotype distributions to that of a large independent trial (N=6,359). As expected, different monitoring modalities tend to provide similar phenotyping information but require the application of technology-dependent conversion factors.

FIGS. 9a and 9b illustrate an example of simultaneous BP data from one patient enrolled in the NCT04548986 trial acquired by an ABPM monitor (Diasys 3 Plus, Novacor, France, FIG. 9a) and the apparatus 10 (Aktiia BP Monitor, Aktiia, Switzerland, FIG. 9b). Note that while the ABPM data was recorded over 24 hours, the data was recorded by the apparatus 10 over one week during and following the ABPM recordings. The monitoring period with the apparatus 10 was extended to one week to account for the day-to-day variability of circadian patterns, and to increase the number of data points registered during daytime and nighttime (because of the lower sampling frequency of the apparatus 10). On the same plot, two estimates of BP dipping are extracted. To calculate the dipping on the ABPM records a common approach of the difference between daytime and night-time BP was used. Daytime and night-time subperiods were defined based on fixed clock-time intervals: 9 am to 9 pm for daytime and 12 am to 6 am for nighttime. Data recorded during the transitional periods were excluded to avoid too much dispersion between individual users. Data points placed at higher distances than the interquartile range from the median value of each subperiod were considered as outliers, and finally, the dip was calculated as the difference between the subperiod medians. To calculate the dipping registered by the apparatus 10, a statistical approach was implemented. Exploiting the fact that the cuffless circadian plot presents a higher density of data points, a parametric model was used to fit the circadian rhythm for SBP and DBP (see continuous “fitting” line in the plot): the night dip was then extracted from one of the model parameters. The estimated night dip for this patient already differs between the two modalities, the ABPM dip (A) appearing to be larger than the Cuffless dip (B)

FIG. 9c shows a statistical analysis of the A>B phenomenon on a preliminary cohort of patients of the NCT04548986 trial. The data from the initial N=20 enrolled patients were processed to estimate the systematic gain difference in the dipping measured by ABPM compared to the dipping measured by the apparatus 10. In this cohort, and after bootstrapping the available samples, we calculated that the ABPM dip is characteristically 3.4 times bigger than the cuffless dip, with a 95% confidence interval ranging from 2.3 to 4.4. It is important to note that the NCT04548986 is not completed, and that a comprehensive analysis of the collected data will be further presented in a dedicated publication.

FIG. 9d shows a further statistical investigation of the same A>B phenomenon, now merging data from an independent ABPM study on N=6,359 patients and real-world data from N=4,644 users of the apparatus 10. According to Kario et al, in a general population one would expect to observe the following distribution of night dipping phenotypes as measured by ABPM measurements (a phenotype is defined as an observable characteristic in the circadian patterns of BP): 16% of individuals are extreme dippers (dipping larger than 20 mmHg), 40% of individuals are normal dippers (dipping between 10 and 20 mmHg), 32% of individuals are non-dippers (dipping between 0 and 10 mmHg) and 12% of individuals are risers (positive dipping). However, when observing the dipping patterns recorded by the apparatus 10, the same distributions are not met, with a clear compression of the dipping distribution. The present analysis consisted of estimating the optimal factor required to expand the dipping distribution of the apparatus 10 to an ABPM-equivalent representation of phenotypes. In this cohort of N=4,644 users, and after bootstrapping, we calculated that the ABPM dip is characteristically 3.1 bigger than the continual cuffless dip, with a 95% confidence interval ranging from 2.8 to 3.4.

By merging real-world data from over 4,000 users of the apparatus 10 with clinical data from a controlled clinical trial, it is demonstrated that the apparatus 10 can estimate patients' BP phenotype. However, because of technological differences between the cuffless technology of the apparatus 10 and the oscillometric ABPM monitors, technology-dependent conversion factors might be needed to compare estimates from both modalities. Table 2 provides a summarized guidance on how to interpret BP phenotyping data from the apparatus 10 when compared to BP characteristics obtained from ABPMs.

The apparatus of the current invention can thus further comprise the step of transforming the calculated cardiovascular values, the calculated cardiovascular parameters, the calculated circadian plots or the calculated physiological parameters into ABPM-equivalent values and plots according to a mapping function. The mapping function can be a pre-calculated affine function like the one presented above (ABPM-like dip can be calculated as 3.1 times the dip calculated by the apparatus 10), or any other type of mapping function pre-calculated from recorded data from a large population, or calculated from any of the user data described in claim 15, or a combination of both. The same mapping approach can also be applied to transform the calculated cardiovascular values, the calculated cardiovascular parameters, the calculated circadian plots or the calculated physiological parameters into HBPM-equivalent values and plots according to a mapping function. The ABPM-equivalent or the HBPM-equivalent can then be used instead of, or in addition to the relevant physiological parameters in the calculation of the cardiovascular risk score of the user.

TABLE 2
Guidance for the interpretation of BP phenotyping data obtained from the
apparatus 10when compared to BP characteristics obtained from ABPM.

| Criterion | ABPM | Apparatus 10 |
|---|---|---|
| Triggering of | Automated, perceived by the | Automated, imperceptible by |
| readings | patient | the patient |
| Frequency of | Every 30 minutes during | On average, approximately |
| readings | daytime, every 1 hour during | one reading per hour |
| | nighttime (might vary across | |
| | devices and guidelines) | |
| Conditions of | Anytime a measurement is | Anytime the patient is quiet or |
| measurement during | triggered and the patient is | performs no important |
| daytime | not performing important | movement (motion tolerance |
| | movements | might vary across devices) with |
| | | no control of body and arm |
| | | position |
| Availability of | Yes | Yes |
| nighttime readings | | |
| Feasibility of long- | No | Yes, even for patients with |
| term monitoring | | reduced compliance |

| | |
|---|---|
| Systematic | SBP night dips of ABPM are ~3.2x larger than those of the |
| difference of | apparatus 10, |
| calculated night | with 95% confidence interval ranging from 2.3 to 4.4 |
| dips | DBP night dips of ABPM are ~2.8x larger than those of the |
| | apparatus 10, |
| | with 95% confidence interval ranging from 1.9 to 3.8 |

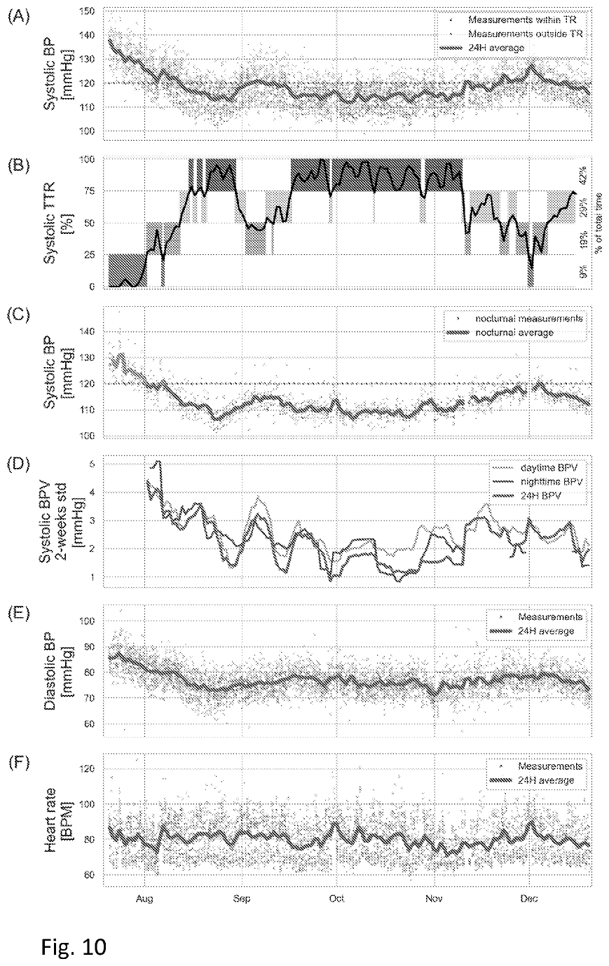

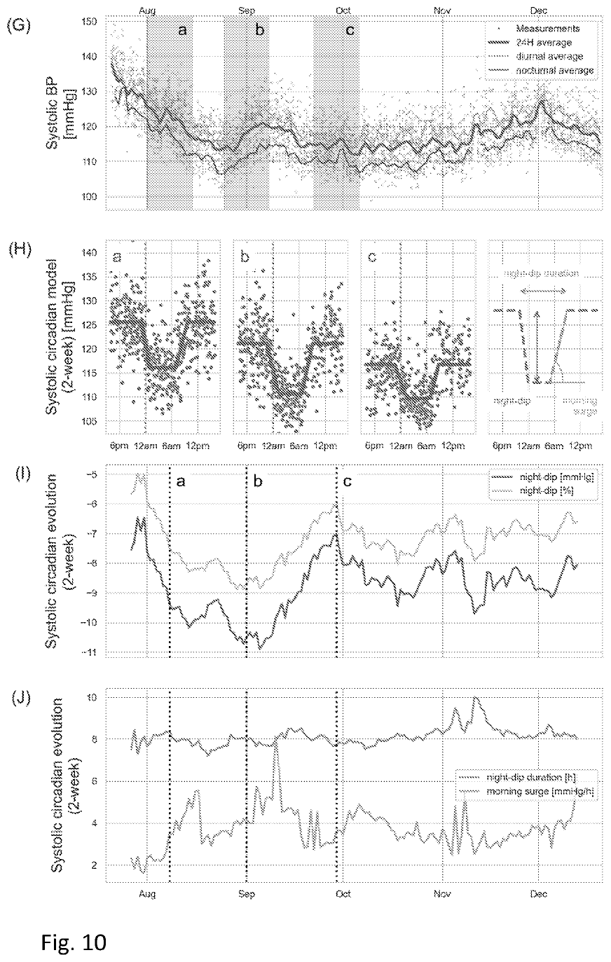

To show the potential the apparatus 10, FIGS. 10a to 10J 5 illustrate a set of new dynamic metrics of BP control estimated on a male patient (51 years old) during five months of continual monitoring by means of the apparatus 10.

In particular, FIGS. 10a to 10J show the new generation of dynamic BP control metrics that can be generated from the data captured 10 from the apparatus 10. The presented time series were captured on a subject over five months, and in addition to standard BP metrics such as 24h, daytime and nighttime averages of SBP, DBP and HR (FIGS. 10a, 10c, 10e, 10f and 10g) it shows novel dynamic metrics such as Time in Therapeutic Range (TTR, FIG. 10b), SBP variability (FIG. 10d), dynamic circadian models (FIG. 10h), dynamic SBP night dips (FIG. 10i) and dynamic night-dip durations and morning surge acceleration (FIG. 10j).

FIG. 10a reports all 4,729 SBP readings performed during the monitoring period, as well as the evolution of the 24 h SBP average. SBP readings within target range (<120 mmHg) are shown as green dots, and outside of target range (>120 mmHg) as red dots. Based on these data, FIG. 10b shows a novel SBP-related metric of BP control: the SBP time in target range (quartiles on Y-axis: 75-100% of time=green, 50-75% of time=yellow, 25-50% of time=orange, and 0-25% of time=red), with the total percent of time spent in each quartile for monitored period on the right side. FIG. 10c further displays the nocturnal average of SBP, with SBP>120 mmHg colored in red, and SBP<120 mmHg colored in green. FIG. 10d displays the mid-term BP variability (SD in mmHg) for daytime (orange), nighttime (black), and average (green). FIG. 10e and FIG. 10f display the diastolic BP average and heart rate 24-hour average (green lines) and all the individual data points (green dots). FIG. 10g displays the SBP daytime (orange), nighttime (black), and 24-hour averages (green). On the same figure, shaded time periods a, b and c correspond to FIG. 10h and are also highlighted in panels I and J. Thus, FIG. 10h shows the SBP circadian patterns at time periods a, b, and c demonstrating for this patient differing patterns of night-dip, night-dip duration, and morning surge. Concerning sleep-related BP parameters, FIG. 10i shows the tracking of the evolution of SBP night dip in mmHg (dark green) and night dip in % (light green). And FIG. 10j shows the tracking of the evolution of the duration of SBP night-dip (red) and quantifies the morning surge (orange).

The advent of 24 h-ABPM demonstrated that the phenotype of BP is more complex than just binary variable (hypertensive or not), and it enabled the demonstration of circadian variations of BP including daytime and nighttime BP components and nocturnal physiological dipping of BP. The predictive value of these different components was compared, and each variable demonstrated predictive value regardless of absolute BP. The average nighttime BP whether systolic or diastolic was a stronger predictor of cardiovascular events. The superior predictive value of average nocturnal BP over 24-hour ABPM, daytime average BP and HBPM has been shown in both hypertensive cohorts and the general population. This strong predictive value is remarkable since the low reproducibility of the dipping pattern has been shown in small studies and more recently in a larger study. Indeed, only a small fraction of hypertensive patients maintain their initial dipping phenotype over 4 years. In addition, the tolerability of ABPM at night is less than during daytime and may hence affect sleep quality. The sleep disturbance induced by cuff inflation has also been shown to affect the association of nighttime ABPM with outcomes.

The apparatus 10 has the potential to overcome these negative features of nighttime ABPM. First, nighttime blood measurement can be repeated over days to months, allowing derivation of a more consistent nighttime phenotype. Second, without any cuff inflation the effect on sleep quality is expected to be insignificant. Nevertheless, these unique characteristics may affect the reference level of normal nighttime cuffless BP, which may need to be newly defined.

To stimulate the exploration of new phenotype-driven assessments of cardiovascular risk, Table 3 provides a list of existing and promising BP phenotypes that can be powered by the large scale deployment the apparatus 10.

TABLE 3
Suggested list of BP phenotypes that can already be identified
via HBPM/ABPM screening, and extended list of BP phenotypes
that will be powered by the deployment of the apparatus 10.

| | |
|---|---|
| Legacy BP phenotypes that can | Sustained normotension |
| already be identified via | White coat hypertension |
| HBPM/ABPM screening | Masked hypertension |
| | Sustained hypertension |
| | Hypotension |
| | Non-dipping/Dipping/Extreme dipping |
| | Short term BP variability |
| Innovative BP phenotypes that | Dynamics of all previous phenotypes |
| will be powered by the | over weeks and months |
| deployment of the apparatus 10 | Working days BP |
| | Weekend days BP |
| | Holiday BP |
| | Dynamic BP response to intervention |
| | Seasonal BP variation |
| | Pregnancy BP variation |
| | Long-term BP variability |

The utility and predictive value of the classic and new phenotypes will have to be demonstrated in longitudinal epidemiological studies before we can ascertain their use in daily practice. Their independent predictive value over average 24-hour or nocturnal BP will be necessary if evidence-based individual treatment is to become a reality. Studying these phenotypes will take time but could certainly provide the physician with a new panel of physiological or provoked BP responses, which may help tailoring the antihypertensive treatment in the future.

The present disclosure further pertains to a non-transitory computer-readable storage medium comprising a computer program product including instructions to cause at least one processor to execute the method for determining a cardiovascular risk score of a user.

1. A non-transitory computer-readable storage medium comprising a computer program product including instructions to cause at least one processor to execute a method comprising:
providing an apparatus configured to measure at least a cardiovascular signal of a user;
measuring said at least a cardiovascular signal during a monitoring period having a duration of at least 48 hours;
the monitoring period being subdivided into more than one monitoring segment, wherein each of said more than one monitoring segment has a duration of 24 hours and comprises a plurality of measurement periods;
determining a cardiovascular value for each measurement period of the plurality of measurement periods within each of said more than at least one monitoring segment;
aggregating the cardiovascular values determined for a given measurement period of each of than one monitoring segment into a cardiovascular parameter cluster;
constructing a circadian plot of 24 hours of the cardiovascular parameter clusters versus the corresponding measurement period for each said more than one monitoring segment-;
calculating a plurality of physiological parameters of the user from the circadian plot; and
calculating a cardiovascular risk score of the user from the determined physiological parameters.

2. The non-transitory computer-readable storage medium method according to claim 1,
wherein the monitoring period has a duration of 48 hours, seven days, one month, or one year.

3. The non-transitory computer-readable storage medium according to claim 1,
wherein the measurement period has a duration of at least 10 second or 30 seconds, namely a duration of 10 seconds, 30 seconds, 1 minute, 5 minutes, one hour, two hours, four hours, or six hours.

4. The non-transitory computer-readable storage medium according to claim 1,
comprising calculating, for each cardiovascular parameter cluster, a cardiovascular representative value of the cardiovascular values aggregated into the cardiovascular parameter cluster.

5. The non-transitory computer-readable storage medium according to claim 4,
wherein calculating the cardiovascular representative value comprises classifying the cardiovascular parameters as inliers or as outliers, and calculating the cardiovascular representative value using only the inlier cardiovascular parameters.

6. The non-transitory computer-readable storage medium according to claim 5,
wherein calculating the cardiovascular representative value comprises classifying the cardiovascular parameters as inliers or as outliers; and
wherein the cardiovascular parameters are weighted according to their probability of being inlier or outlier.

7. The non-transitory computer-readable storage medium according to claim 1,
wherein determining the plurality of physiological parameters of the user comprises a step of fitting a model to the circadian plot, the physiological parameters corresponding to parameters of the model.

8. The non-transitory computer-readable storage medium according to claim 7,
wherein the model is one of a linear model, fitted via a least-square optimization, a non-linear model, a constrained model, an unconstrained model.

9. The non-transitory computer-readable storage medium according to claim 1,
wherein the physiological parameters include any one of parameters: diurnal BP value, absolute night-dipping amplitude, temporality of the dipping start, duration of the pre-nocturnal ramp, duration of the dipping plateau, or duration of the post-nocturnal ramp.

10. The non-transitory computer-readable storage medium according to claim 7,
wherein the model further uses non-physiological parameters.

11. The non-transitory computer-readable storage medium according to claim 1,
further comprising combining at least two of the physiological parameters to obtain one or a plurality of relevant physiological parameters.

12. The non-transitory computer-readable storage medium according to claim 11,
wherein said one or a plurality of relevant physiological parameter includes any one of: daytime BP value, nocturnal BP value, relative night-dipping amplitude, complete night-dipping duration, time in target range (TTR), BP variability patterns, slope of the morning surge, night-time SBP, night-time DBP, night-time HR, SBP dip, DBP dip, HR dip, SBP morning surge, DBP morning surge, HR morning surge, SBP dipping duration, DBP dipping duration, HR dipping duration, SBP/DBP/HR synchronization, responses to types of medications, BP medication adherence, patient engagement measures, or responses to lifestyle interventions.

13. The non-transitory computer-readable storage medium according to claim 11,
wherein the relevant physiological parameters include a blood pressure phenotype of the user.

14. The non-transitory computer-readable storage medium according to claim 13,
wherein the relevant physiological parameters include any one of: true normotension, white-coat hypertension, masked hypertension, sustained hypertension, hypotension, night-dipping, night-raising, or phenotypes that predict responses to specific medications or therapies.

15. The non-transitory computer-readable storage medium according to claim 1,
wherein calculating the cardiovascular risk score of the user comprises using user data.

16. The non-transitory computer-readable storage medium according to claim 15,
wherein user data include any one of: as age, weight, height, gender, ethnicity, lipid levels, diabetes status, smoking, CT calcium (Agatston score), family history, genetic markers of risk, actigraphy information, workout information, dietary information, stress level, general feeling, hormonal data, menstrual cycle information, medication intake, working-day/week-end information, seasonal information, sleep quality information, go to bed patterns or any of the parameters used in the calculation of cardiovascular risk score in clinical guidelines such as the ACC/AHA guidelines, the ESC guidelines, or the MESA database or any of the non-physiological parameters.

17. The non-transitory computer-readable storage medium according to claim 1,
wherein the cardiovascular risk score is any of 10-year risk of cardiovascular disease, 10-year risk of heart disease, 10-year risk of stroke or any other clinically relevant cardiovascular risk score.

18. The non-transitory computer-readable storage medium according to claim 1,
wherein the cardiovascular parameter is at least any of a blood pressure value, a heart rate value, a cardiac output value, a blood glucose value, a measure of physical activity, measures of sleep quantity and quality, an electrocardiogramanal, a photoplethysmographic signal, a bioimpedance signal, or an ultra-sound signal.

19. An apparatus for determining a cardiovascular risk score of a user comprising:
a measuring module configured to measure a cardiovascular signal of the user during a monitoring period having a duration of at least 48 hours, wherein the monitoring period is subdivided into more than one monitoring segments, each of said more than one monitoring segment having a duration of twenty-four hours and comprising a plurality of measurement periods;
a processor configured to determine a cardiovascular value for each measurement period of the plurality of measurement periods within each of said more than one monitoring segment, the processor being further configured to aggregate the cardiovascular values determined for a corresponding measurement period of each of said more than one monitoring segment into a cardiovascular parameter cluster, the processor being further configured to construct a circadian plot of twenty-four hours of the parameter clusters versus the corresponding measurement period for each said more than one monitoring segment, using the circadian plot to determine a plurality of physiological parameters of the user from the circadian plot, and using the determined physiological parameters to calculate a cardiovascular risk score of the user;
the apparatus further comprising an interface to display and/or transmit the calculated cardiovascular risk score.

20. The apparatus according to claim 19,
wherein the interface comprises an application on a smartphone, a tablet, a computer, a smartwatch or any portable device.

21. The apparatus according to claim 19, being connectable to via any of a wired or wireless connections, including WiFi or Bluetooth or cellular supports.

22. (canceled)
